(19) United States
(12) Patent Application Publication
(10) Pub. No.: US 2010/0291663 A1
(43) Pub. Date: Nov. 18, 2010

(54) CULTURE APPARATUS

(75) Inventor: Ryuji KOSHIBA, Yokohama-shi (JP)

Correspondence Address: OLIFF & BERRIDGE, PLC, P.O. BOX 320850, ALEXANDRIA, VA, 22320-4850, US

(73) Assignee: NIKON CORPORATION, Tokyo (JP)

(21) Appl. No.: 12/836,180

(22) Filed: Jul. 14, 2010

Related U.S. Application Data
(63) Continuation of application No. PCT/JP2009/050800, filed on Jan. 21, 2009.

(30) Foreign Application Priority Data
Jan. 21, 2008 (JP) 2008-010110

(51) Int. Cl. C12M 1/36; C12M 1/34; C12M 1/00

(52) U.S. Cl. 435/286.1; 435/287.1; 435/289.1

(57) ABSTRACT

The present invention relates to a culture apparatus that enables cells to be observed and dispensed without damaging the cells. An internal space of an incubator unit is provided with a dispensing area for arranging dispensers for dispensing into a culture vessel, an observation area for arranging an observation unit for observing a sample placed in the culture vessel through an observation optical system, and a transport area for arranging a transport unit for transporting the culture vessel in the horizontal and vertical directions, the dispensing area and the observation area are respectively arranged adjacent to the transport area in the horizontal direction which is a direction of transport of the transport unit, and the transport unit transports the culture vessel between the dispensing area and the observation area. The present invention can be applied to a cell culture apparatus that houses a culture vessel containing a sample in an internal space and cultures the sample under prescribed environmental conditions.

### TECHNICAL FIELD

The present invention relates to a culture apparatus, and more particularly, to a culture apparatus that enables observation and dispensing of cells without causing damage to the cells by not allowing the cells to be outside the culture environment.

### BACKGROUND ART

Within an incubator environment for culturing and observing cells, the addition of reagents or replacement of media requires the culture vessel to be removed and transferred to a special-purpose dispensing apparatus separate from the cell culture observation apparatus provided with an observation function.

Patent Document 1 proposes a technology for transporting a culture vessel to a culture chamber or processing chamber, for which the environment thereof is individually controlled, using various types of transport mechanisms such as a transport robot, handling robot or conveyor.

[Patent Document 1] Japanese Patent Application Laid-open No. 2004-350641

### BRIEF DESCRIPTION OF THE DRAWINGS

FIG. 1 is a front view showing the overall configuration of a cell culture apparatus to which the present invention is applied;

FIG. 2 is an overhead view showing the configuration of a cell culture apparatus;

FIG. 3 is a drawing showing the detailed configuration of a tray;

FIG. 4 is a drawing showing the detailed configuration of a tip solution storage vessel;

FIG. 5 is a drawing showing the detailed configuration of a transport unit;

FIG. 6 is a schematic drawing depicting dispensing work;

FIG. 7 is a schematic drawing depicting dispensing work;

FIG. 8 is a schematic drawing depicting dispensing work;

FIG. 9 is a drawing representing the flow of attaching a dispenser tip;

FIG. 10 is a schematic drawing depicting dispensing work;

FIG. 11 is a drawing representing the flow of removing a dispenser tip; and

FIG. 12 is a flow chart for explaining processing for determining whether or not dispensing work is to be performed.

### EXPLANATION OF REFERENCE NUMERALS

1 cell culture apparatus, 11 incubator unit, 11A access door, 11B inner door, 12 stand unit, 13 control box, 14 personal computer, 21 stocker unit, 22 transport unit, 22A stage base, 22B Y stage, 22C guide shaft, 22D drive shaft, 22E motor, 22F Z stage, 22G drive shaft, 22H motor, 22I drive unit, 22J dispensing stage, 22K arm, 22L₁ and 22L₂ dispenser, 22M rotating shaft, 23 lid opening and closing unit, 24A and 24B dispensing unit, 25 observation unit, 25A illumination unit, 25B CCD camera, 26 carrier unit, 27 pump unit, 28 tube, 31 tray, 41 culture vessel, 42 tip solution storage vessel, 42A vessel portion, 42A₁ unused dispenser tip area, 42A₂ used dispenser tip area, 42A₃ solution area, 42A₄ waste liquid area, 42B lid portion, 51 dispenser tip, 52 hole, 53 aspiration port, 54 waste liquid port, 61A and 61B tray holder, 62A and 62B tray lug locking portion

### BEST MODE FOR CARRYING OUT THE INVENTION

The following provides an explanation of embodiments of the present invention with reference to the drawings.

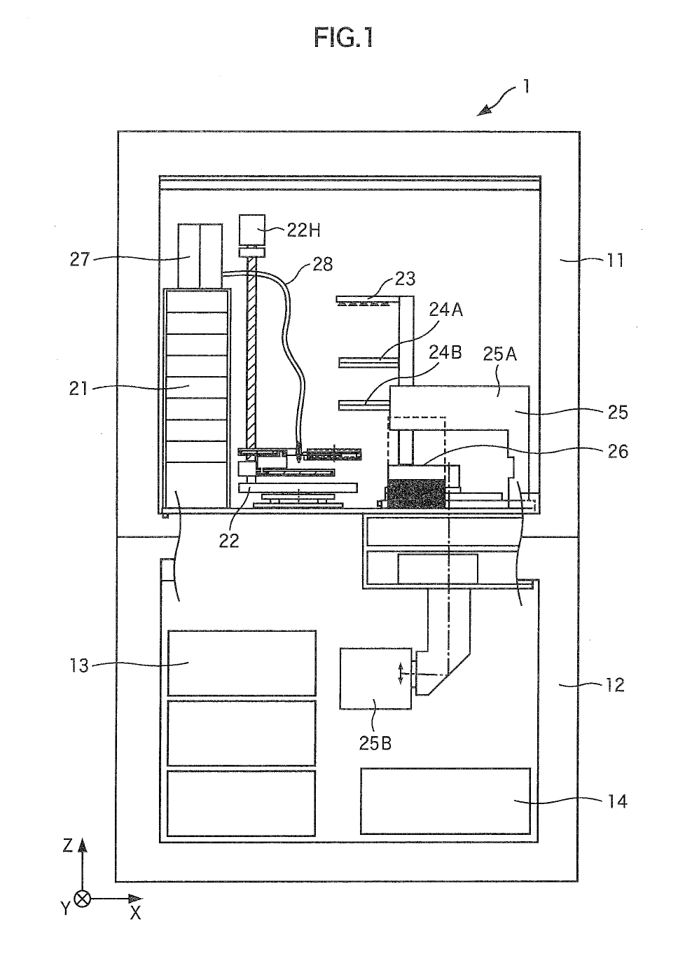

FIG. 1 is a front view showing the overall configuration of a cell culture apparatus to which the present invention is applied.

In the example of FIG. 1, a cell culture apparatus 1 is composed of an incubator unit 11 and a stand unit 12 arranged on the lower side thereof. A temperature control mechanism composed of a temperature control device and the like that uses a heater, a temperature control mechanism composed of a spraying device and the like that that sprays a mist, a gas control mechanism composed of a gas induction unit and the like connected to an external carbon dioxide tank, or an environmental sensor and the like that detects a cell culture environment of an internal space (none of which are shown in the drawings), for example, is provided within the incubator unit 11. In addition, the inside of the incubator unit 11 is covered with a thermally insulating material. As a result, the inside of the incubator unit 11 is sealed to maintain the cell culture environment during cell culturing, and by maintaining at a constant temperature by circulating air, for example, the cell culture environment is maintained at, for example, a temperature of 37° C., humidity of 90% and carbon dioxide concentration of 5%.

In addition, as shown in FIG. 1, a control box 13 and personal computer 14 and the like for controlling each unit of the cell culture apparatus 1 are housed within the stand unit 12 on which the incubator unit 11 is placed.

A stocker unit 21, transport unit 22, lid opening and closing unit 23, dispensing units 24A and 24B, an observation unit (microscope unit) 25 and a carrier unit 26 are installed within the incubator unit 11.

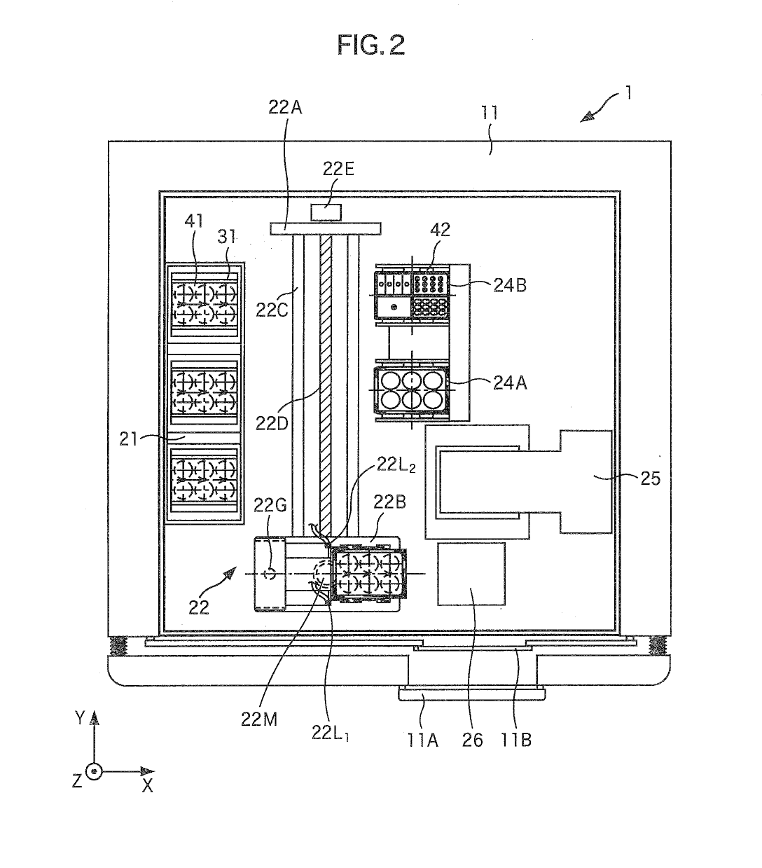

The following provides an explanation of the layout and configuration of each mechanism provided within the incubator unit 11 with reference to the overhead view of the cell culture apparatus 1 of FIG. 2.

The stocker unit 21 is the location where a culture vessel 41 and tip solution storage container 42 are stored placed on trays 31. Namely, the culture vessel 41 and the tip solution storage container 42 can be housed in the stocker unit 21 on individual trays 31.

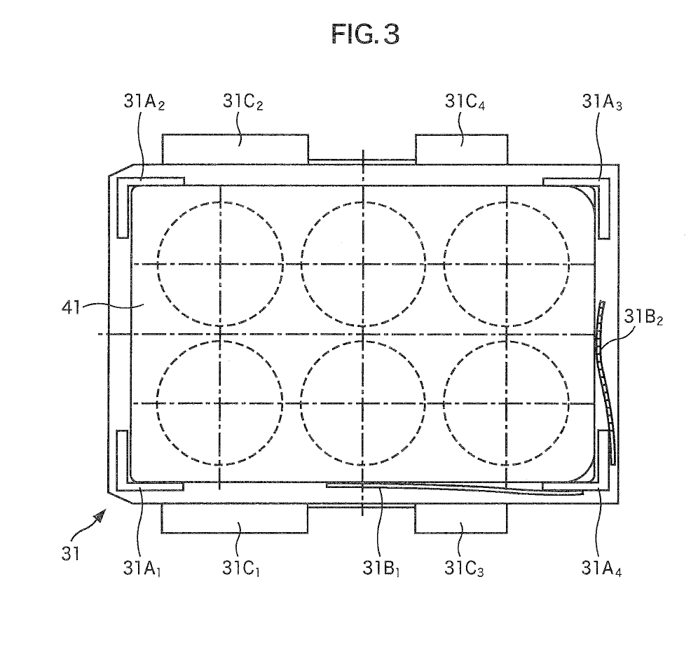

A detailed configuration of the tray 31 is as shown in FIG. 3. Namely, as shown in the overhead view of the tray 31 of FIG. 3, the tray 31 is composed of a shape that is capable of carrying the culture vessel 41 or the tip solution storage vessel 42, and further has fixing blocks 31A₁ to 31A₄ and springs 31B₁ and 31B₂ (to be simply referred to as the fixing blocks 31A and the springs 31B). In the example of FIG. 3, a culture vessel 41 is fixed by the fixing blocks 31A and the springs 31B. In addition, the tray 31 is provided with lugs 31C₁ to 31C₄ (to be simply referred to as the lugs 31C), and the transport unit 22 transports the tray 31 by carrying the lugs 31C.

In addition, although the example of FIG. 3 shows the culture vessel 41 placed on the tray 31, the tray 31 can also carry the tip solution storage vessel 42 that has a shape that enables it to be placed on the tray 31. For example, the tip solution storage vessel 42 has the same shape or similar shape as that of the culture vessel 41.

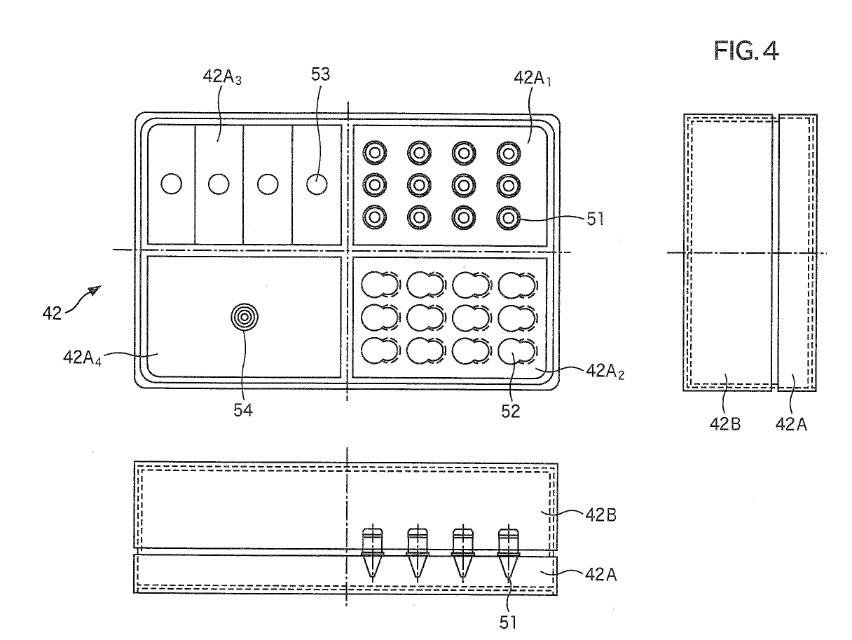

A detailed configuration of the tip solution storage vessel 42 is as shown in FIG. 4. Namely, as shown by the three views (front view, overhead view and side view) of the tip solution storage vessel 42 of FIG. 4, the tip solution storage vessel 42 is composed of a vessel portion 42A and a lid portion 42B, and in the case of not using the tip solution storage vessel 42, the lid portion 42B is not placed over the vessel portion 42A, and when the tip solution storage vessel 42 is used, the lid portion 42B is removed from the vessel portion 42A.

The vessel portion 42A has four areas consisting of an unused dispenser tip area 42A₁, a used dispenser tip area 42A₂, a solution area 42A₃ and a waste liquid area 42A₄.

Unused dispenser tips 51 for attaching to a disperser 22L₁ of the transport unit 22 in a clean bench and the like are placed in the unused dispenser tip area 42A₁. Although a maximum of 12 dispenser tips 51 can be placed the unused dispenser tip area 42A₁ in the case of the example shown in FIG. 4, the number thereof is not limited to 12. Furthermore, details of the method used to attach the unused dispenser tips 51 to the dispenser 22L₁ will be described hereinafter.

Used dispenser tips 51 are discarded in the used dispenser tip area 42A₂ by using a hole 52. Although 12 holes 52 are provided in the example of FIG. 4, the number of thereof is not limited to 12. Furthermore, details of the method used to discard the used dispenser tips 51 will be described hereinafter.

In addition, a solution such as a reagent to be subsequently used is placed in the solution area 42A₃, and is aspirated from an aspiration port 53 by the dispenser 22L₁. Furthermore, although an embodiment in which four types of solutions can be placed is shown in the example of FIG. 4, the number of types of solutions is not limited thereto, but rather the number of thereof may be changed corresponding to the amounts and types of reagents used. In addition, although reagents used may be transported within the apparatus after preliminary warming to a temperature at which they are to be used as necessary, reagents can also be placed in the apparatus in anticipation of the time during which they are warmed.

Used waste liquid flows into the waste liquid area 42A₄. More specifically, since the end of the dispenser 22L₁ is inserted into a waste liquid port 54 in the waste liquid area 42A₄, waste liquid expelled from the dispenser 22L₁ accumulates therein.

As has been described above, the tip solution storage vessel 42 is provided with respective areas for storing each type of element required to realize dispensing work, and the transport unit 22 places the tip solution storage vessel 42 on the tray 31 and transports to each mechanism within the incubator unit 11.

Furthermore, although the four areas consisting of the unused dispenser tip area 42A₁ to the waste liquid area 42A₄ correspond to each region of the vessel portion 42_{A} divided into four equal parts in the example of FIG. 4, these regions are not necessarily required to be divided equally, but rather the size of a region of a certain area can be increased or decreased. For example, in the case the usage frequency of the unused dispenser tip area 42A₁ is high, the size of that region can be increased while the sizes of the regions of the other areas can be correspondingly decreased.

In addition, although the explanation of the example of FIG. 4 uses the vessel portion 42A having four areas consisting of the unused dispenser tip area 42A₁ to the waste liquid area 42A₄, a configuration may also be employed in which, for example, the vessel portion 42A only has two areas consisting of the unused dispenser tip area 42A₁ and the and used dispenser tip area 42A₂, or a configuration in which the vessel portion 42A only has two areas consisting of the solution area 42A₃ and the waste liquid area 42A₄. In short, the vessel portion 42A is only required to have a number of areas among the four areas of the unused dispenser tip area 42A₁ to the waste liquid area 42A₄ corresponding to the type of dispensing work carried out within the incubator unit 11. In addition, although the dispenser tips are indicated as being disposable tips in the present embodiment, the dispenser tips may also be washed.

The tip solution storage vessel 42 of the present embodiment is at least composed of the vessel portion (vessel body portion) 42A in which a plurality of compartments are formed, a first area (storage region) 42A₁ for storing and retaining unused dispenser tips 51 formed in one of the compartments of the vessel portion 42A, and a second area (recovery region) 42A₂ for recovering and storing used dispenser tips 51 formed in another compartment of the vessel portion 42A.

Moreover, in addition to the above-mentioned configuration, the tip solution storage vessel 42 has the solution storage area 42A₃ for housing a solution such as a reagent aspirated by the dispenser tips 51 in another compartment of the vessel portion 42A.

A configuration is employed in which a plurality of unused dispenser tips 51 are housed in the first area 42A₁, and each tip is held while orderly arranged in the same direction in the form of a matrix. In addition, the second area 42A₂ has at least one hole 52 formed therein, the hole 52 has a portion having an inner diameter such that a protruding ring (or cylindrical indentation) of the sidewall of a tip catches thereon, and a chamber that houses used dispenser tips 51 that have caught on the hole 52 and been released is formed below the hole 52.

In this manner, the tip solution storage vessel 42 is composed by having areas demonstrating a plurality of functions integrally formed within a single rectangular vessel. By providing a plurality of this tip solution storage vessel 42, a user is able to easily replace dispenser tips with new ones or recover used tips.

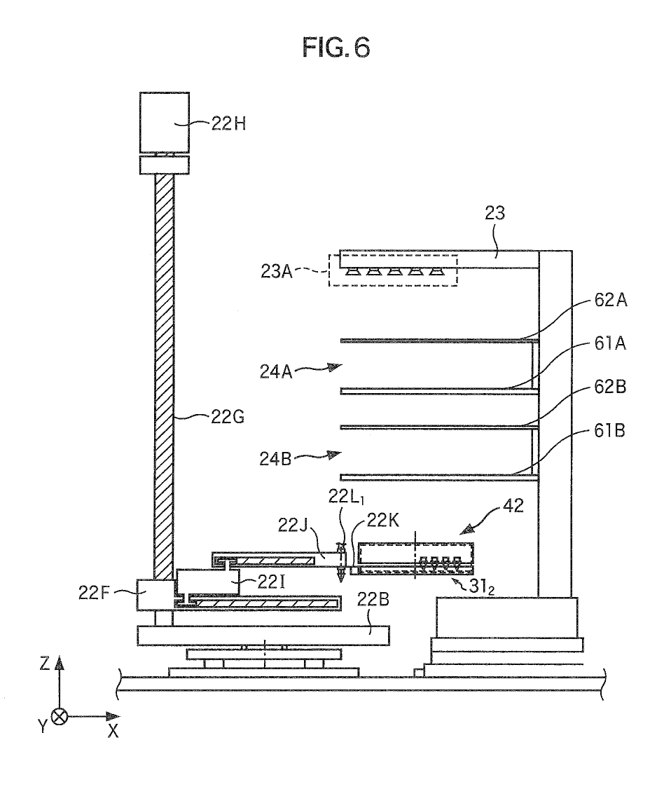

Returning to FIG. 2, the transport unit 22 is a mechanism that transports the culture vessel 41 or the tip solution storage vessel 42 placed on the tray 31 housed in the stocker unit 21 to any of the lid opening and closing unit 23, the dispensing unit 24A, dispensing unit 24B or observation unit 25. Namely, the transport unit 22 supports the tray 31 carrying the culture vessel 41 or the tip solution storage vessel 42 to each mechanism, together with moving the tray 31 to and from the stocker unit 21. Furthermore, although the lid opening and closing unit 23 is not shown in the overhead view of FIG. 2 in order to more readily illustrate the dispensing unit 24A and the dispensing unit 24B, the lid opening and closing unit 23 is installed above the dispensing units 24A and 243 as is clear by referring to FIG. 1 and other subsequently described drawings such as FIG. 6.

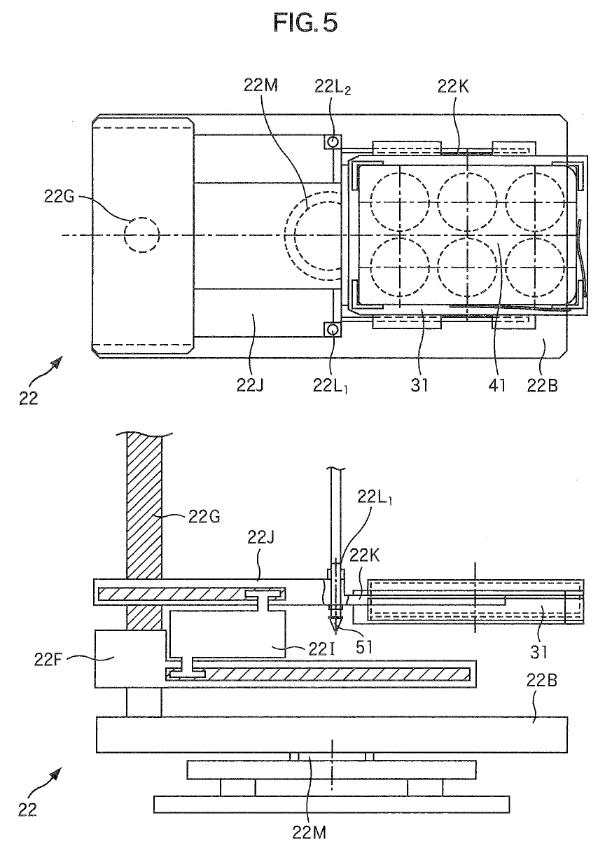

The following provides an explanation of the detailed configuration of the transport unit 22 with reference FIGS. 1 and 2 as well as the enlarged view of the transport unit 22 of FIG. 5. Furthermore, in FIG. 5, the upper drawing represents an overhead view of the transport unit 22, while the lower drawing represents a front view of the transport unit 22.

As shown in FIGS. 1 and 2, dispensing areas containing the dispenser units 24A and 24B that carrying out dispensing into a culture vessel, an observation area for arranging the observation unit 25 for observing samples placed in a culture vessel by means of an observation optical system, and a transport area for arranging the transport unit 22 for transporting a culture vessel in the horizontal and vertical directions are provided in the internal space of the incubator unit 11 of the cell culture apparatus 1. In this internal space, the dispenser area and the observation area are respectively arranged adjacent to the transport area in the horizontal direction which is a direction of transport of the transport means, and the transport unit 22 transport culture vessels between the dispensing area and the observation area in order to carry out dispensing work in the dispensing area and observation work in the observation area.

In FIG. 2, a Y stage 22B is attached to a stage base 22A through a guide shaft 22C and a drive shaft 22D for the Y axis. The Y stage 22B moves in the direction of the Y axis by rotation of a motor 22E. Furthermore, the stage base 22A is fixed to a case.

A Z stage 22F is attached to the Y stage 22B through a drive shaft 22G for the Z axis. The Z stage 22F moves in the direction of the Z axis by rotation of a motor 22H. A dispensing stage 22J is attached to the Z stage 22F through a drive unit 22I. The dispensing stage 22J is a stage provided in the uppermost portion of the transport unit 22, and moves in the direction of the X axis as a result of being driven by the drive unit 22I. In other words, as shown in FIG. 5, the drive unit 22I is, for example, a rack and pinion mechanism in which pinions are respectively provided on the sides attached to both the Z stage 22F and the dispensing stage 22J, and is attached to the Z stage 22F and the dispensing stage 22J as a result of these pinions respectively engaging with racks formed on the two stages. As a result, the dispensing stage 22J can be slid by a prescribed amount in the direction of the X axis by being driven by the drive unit 22I.

Two dispensers in the form of the dispenser 22L₁ and a dispenser 22L₂ are attached to the dispensing stage 22J. The dispenser 22L₂ is for aspirating and adding a solution such as a reagent, while the dispenser 22L₂ is for aspirating waste liquid. More specifically, the dispenser 22L₁ and the dispenser 22L₂ are respectively connected to a pump unit 27 (FIG. 1) through tubes 28, and solution is aspirated and added by the dispenser 22L₁ by being driven by the pump unit 27 or waste liquid is aspirated by the dispenser 22L₂. In addition, the dispenser tips 51 respectively attached to the dispenser 22L₁ and the dispenser 22L₂ can be attached and removed.

Furthermore, the dispenser units are collectively explained as dispensers 22L in cases in which it is not necessary to distinguish the two dispensers.

An arm 22K is also removably attached to the dispensing stage 22J on an end thereof. The lugs 31C of the tray 31 are placed on the arm 22K. Namely, the arm 22K is used as a transfer member during transport and placement of the tray 31. Furthermore, a member having a large coefficient of friction (such as rubber) is preferably affixed to the contact surfaces of the arm 22K and the tray 31.

In addition, the Y stage 22B rotates about a rotating shaft 22M within the XY plane by rotation of a rotary motor (not shown). Namely, in addition to the transport unit 22 transporting to mechanisms on the right side of the transport unit 22 in FIG. 2, such as the lid opening and closing unit 23 (not shown in FIG. 2), the dispenser unit 24A, the dispenser unit 24B and the observation unit 25, by turning the Y stage 22B 180 degrees and facing in the opposite direction, the transport unit 22 is also able to transport to mechanisms on the left side of the transport unit 22 in FIG. 2, such as the stocker unit 21. Incidentally, when the Y stage 22B is rotated, the dispensing stage 22J is preferably reduced in size on the side of the stage 22F to decrease the radius of rotation.

In this manner, the transport unit 22 composed as described above allows the tray 31 supported by the arm 22K to move in the three directions of X, Y and Z axes, and is also able to rotate 180 degrees about the Z axis. As a result, the transport unit 22 is able to support and transport the tray 31 to each mechanism within the incubator unit 11, while also being able to move the tray 31 to and from the stocker unit 21.

In addition, the transport unit 22 transports the storage vessel 41 placed on the tray 31 to the observation unit 25 in the case of observing the culture vessel 41 with the observation unit 25.

The observation unit 25 is mainly composed of an illumination system and an observation system, and a portion thereof is also housed in the stand unit 12 in addition to being housed within the incubator unit 11. In the illumination system, light from a light source such as a light-emitting diode (LED) in the form of an illumination unit 25A enters an observation stage through a phase ring or condenser lens after undergoing rectangular collimation. The light then enters a sample within the culture vessel 41 placed on the tray 31, which has been transported to a space within the observation stage by the transport unit 22, in the form of illumination light. The sample illuminated by light from the illumination system generates light corresponding to the culturing status thereof. Light generated from the sample in the direction of transmission is guided to the observation system after passing through the observation stage.

In the observation system, light generated from the sample in the direction of transmission enters a charge coupled device (CCD) camera 25B after passing through an object lens, an intermediate zoom lens, fluorescent illumination unit and internal lenses of the CCD camera 25B and the like. At this time, an image of the sample is formed on the imaging surface of the CCD camera 25B by an imaging optical system. An image captured by the CCD camera 25B is displayed on, for example, a monitor (not shown).

The cell culture apparatus 1 is composed in the manner described above.

With respect to the method used to transport the culture vessel 41 and the tip solution storage vessel 42 inside the incubator unit 11, after placing the dispenser tips 51 in the unused dispenser tip area 42A₁ of the vessel portion 42A of the tip solution storage vessel 42, and placing reagent in the solution area 42A₃ followed by covering with the lid portion 42B in a clean bench, the tip solution storage vessel 42 is placed on a tray 31₂. In addition, the culture vessel 41 is placed on a tray 31₁ different from the tray 31₂ on which the tip solution storage vessel 42 has been placed.

Furthermore, although the trays 31 on which the culture vessel 41 and the tip solution storage vessel 42 are placed are explained by referring to as the tray 31₁ and the tray 31₂, respectively, in order to distinguish the trays 31 on which the culture vessel 41 and the tip solution storage vessel 42 are placed, they are simply referred to as the trays 31 in cases in which it distinction thereof is not particularly necessary. In addition, the culture vessel 41 and the tip solution storage vessel 42 (vessel portion 42A thereof) are respectively fixed by the fixing blocks 31A and the springs 31B of the trays 31 on which they are placed.

Next, the tray 31₁ on which the culture vessel 41 is placed and the tray 31₂ on which the tip solution storage vessel 42 is placed are placed on a carrier that collectively houses a plurality of the trays 31. An access door 11A and an inner door 11B of the incubator unit 11 are then opened by the operation of an operator, and a carrier housing the plurality of trays 31 is placed on the carrier unit 26 within the incubator unit 11 (the dotted line of the carrier unit 26 in FIG. 1 represents a carrier placed thereon). Whereupon, the trays 31 placed on the carrier are transported to a prescribed location within the incubator unit 11 by the transport unit 22 that operates according to an operation by the operator. For example, in the case of immediately using the tip solution storage vessel 42, the tray 31₂ on which the vessel is placed is transported to the dispenser unit 24B, and in the case of using after warming for a predetermined amount of time, the tray 31₂ on which the vessel is placed is transported to the stocker unit 21 and housed therein. In addition, the tray 31₁ on which the culture vessel 41 is placed, for example, is transported to the stocker unit 21 and housed therein at this time.

As a result, prior to the start of sample culturing, the tray 31₁ on which the culture vessel 41 is placed and the tray 31₂ on which the tip solution storage vessel 42 is placed are transported by the transport unit 22 and housed in each level of the stocker unit 21, the dispenser unit 24B and the like.

Furthermore, in the present embodiment, each level of the stocker unit 21 may be composed to accommodate individual trays 31, or each level of the stocker unit 21 may be composed corresponding to the size of the carrier that collectively carries a plurality of the trays 31. In addition, the stocker unit 21 is installed along a sidewall within the case of the incubator unit 11.

However, as was previously described, in addition to transporting the trays 31 on which the culture vessel 41 and the tip solution storage vessel 42 are placed to each mechanism within the incubator unit 11, since the transport unit 22 also has the dispensers 22L, it is also able to place solution in the culture vessel 41 in the dispenser unit 24A.

Therefore, the following provides an explanation of dispensing work carried out by the transport unit 22, or in other words, the flow of work consisting of transporting the tip solution storage vessel 42 to the dispensing unit 24B, aspirating solution from the tip solution storage vessel 42, and adding to the culture vessel 41 transported to the dispensing unit 24A, with reference to FIGS. 6 to 11.

Furthermore, the locations of each insertion hole of the four areas of the unused dispenser tip area 42A₁, the used dispenser tip area 42A₂, the solution area 42A₃ and the waste liquid area 42A₄ within the tip solution storage vessel 42 when the trays 31₂ on which the tip solution storage vessel 42 is placed are fixed to the dispenser unit 24B are registered in advance in the form of coordinates corresponding to those locations.

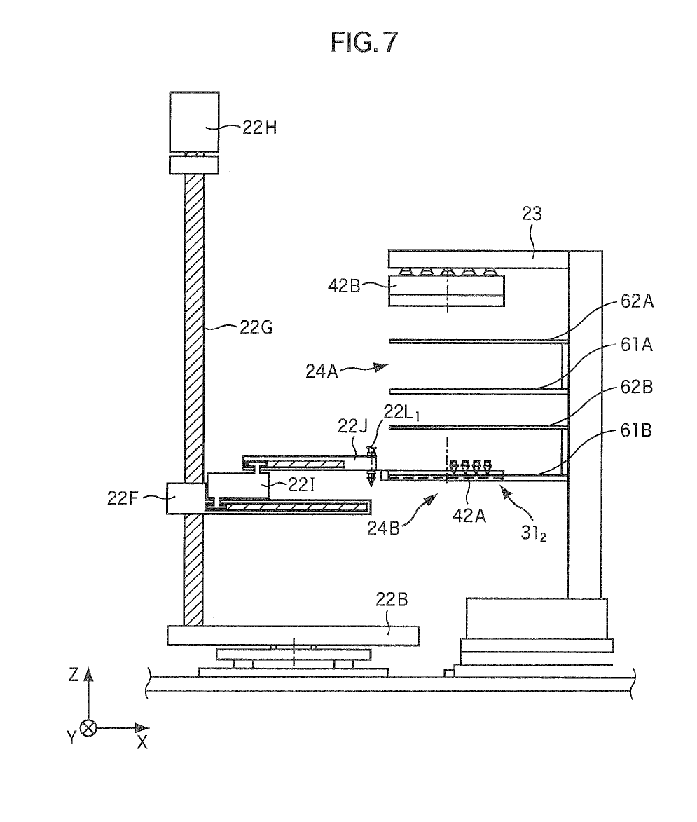

First, as shown in FIG. 6, the tip solution storage vessel 42 placed on the tray 31₂ is transported to an area where the lid opening and closing unit 23 is installed by the transport unit 22. Furthermore, FIG. 6 depicts the state when the transport unit 22 has moved to the area where the lid opening and closing unit 23, the dispensing unit 24A and the dispensing unit 24B are installed within the incubator unit 11. In addition, similar states are depicted in FIGS. 7, 8 and 10 to be subsequently described.

At this time, since the tip solution storage vessel 42 is transported directly beneath a suctioning portion 23A, the transport unit 22 causes the suctioning portion 23A to contact the tip solution storage vessel 42 by further moving the tip solution storage vessel 42 in the positive direction of the Z axis. Whereupon, as shown in FIG. 7, only the lid portion 42B of the vessel portion 42A and the lid portion 42B that compose the tip solution storage vessel 42 is vacuum-suctioned by the suctioning portion 23A. In other words, in the transport unit 22, the lid portion 42B is removed and only the vessel portion 42A is supported by the arm 22K while placed on the tray 31₂.

Next, as shown in FIG. 7, the transport unit 22 transports the tip solution storage vessel 42 from which the lid portion 42B has been removed, namely the vessel portion 42A, to a tray holder 61B of the dispensing unit 24B by moving in the negative direction of the Z axis. Furthermore, at this time, the lugs 31C of the tray 31₂ placed on the tray holder 61B are clamped and fixed by the tray holder 61B and a tray lug locking portion 62B of the dispensing unit 24B by moving the tray lug locking portion 62B in the negative direction of the Z axis. Namely, when carrying out dispensing work, the lugs 31C of the tray 31₂ are fixed by the tray lug locking portion 62B at the start of dispensing when the dispensing work mode has been switched on. Subsequently, once dispensing work has been completed, the dispensing work mode is switched off, and the tray lug locking portion 62B that had been fixing the lugs 31C of the tray 31₂ is disengaged by driving in the positive direction of the Z axis, thereby freeing the lugs 31C of the tray 31₂.

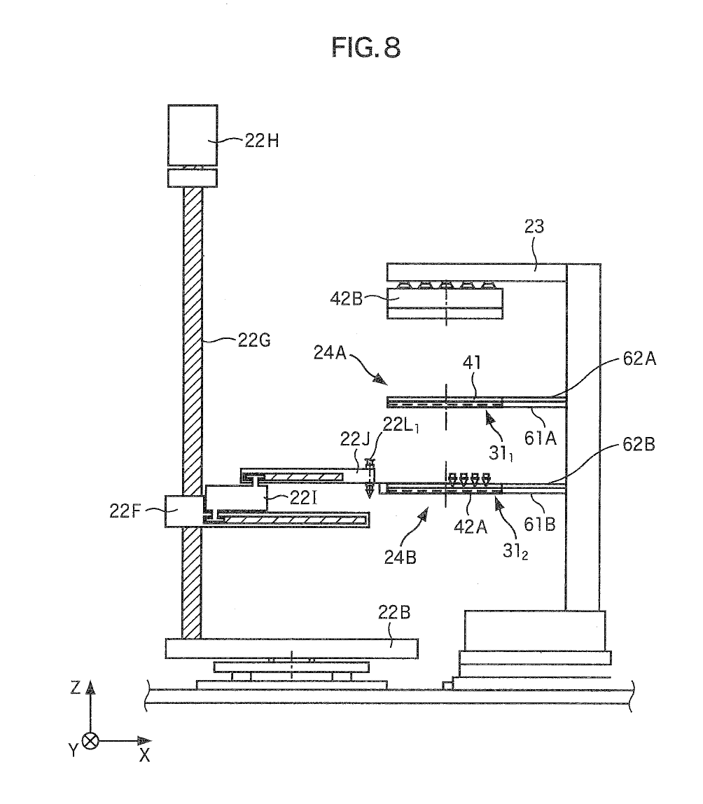

Subsequently, the transport unit 22 places the culture vessel 41 on the tray 31₁ and transports it to the tray holder 61A of the dispensing unit 24A in the same manner as when transporting the tip solution storage vessel 42. As shown in FIG. 8, the lugs 31C of the tray 31₁ placed on the tray holder 61A are fixed by a tray lug locking portion 62A of the dispenser unit 24A. Furthermore, in the case the culture vessel 41 is also provided with a lid (lid portion), the lid portion of the culture vessel 41 is suctioned by the suctioning portion 23A of the lid opening and closing unit 23 in the same manner as in the case of removing the lid portion 42B of the tip solution storage vessel 42. In this case, the lid opening and closing unit 23 is respectively installed above, for example, the tray holder 61A and the tray holder 61B, and removes each of the lid portions from the culture vessel 41 and the tip solution storage vessel 42.

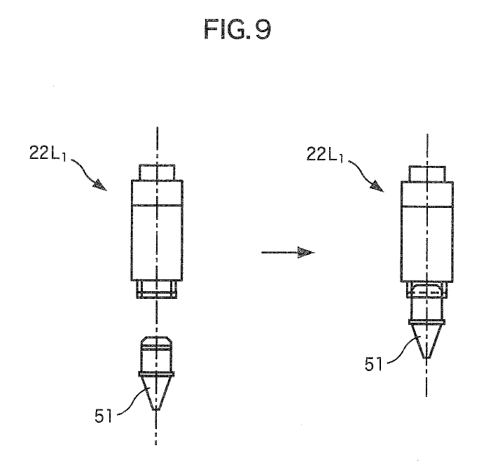

As shown in FIG. 8, the transport unit 22 then moves the dispenser 22L₁ to a location corresponding to above the unused dispenser tip area 42A₁ of the vessel portion 42A fixed by the tray lug locking portion 62A that has been transported to the lower level of the tray holder 61B. The transport unit 22 attaches a dispenser tip 51 placed in an insertion hole in the unused dispenser tip area 42A₁ to the dispenser 22L₁ based on preliminary registered coordinate information for each insertion hole. With respect to the method used to attach the dispenser tip 51, as shown on the left side of FIG. 9, for example, the transport unit 22 presses the dispenser 22L₁ onto the dispenser tip 51 placed in an insertion hole to attach the dispenser tip 51 to the dispenser 22L₁ as shown on the right side of FIG. 9.

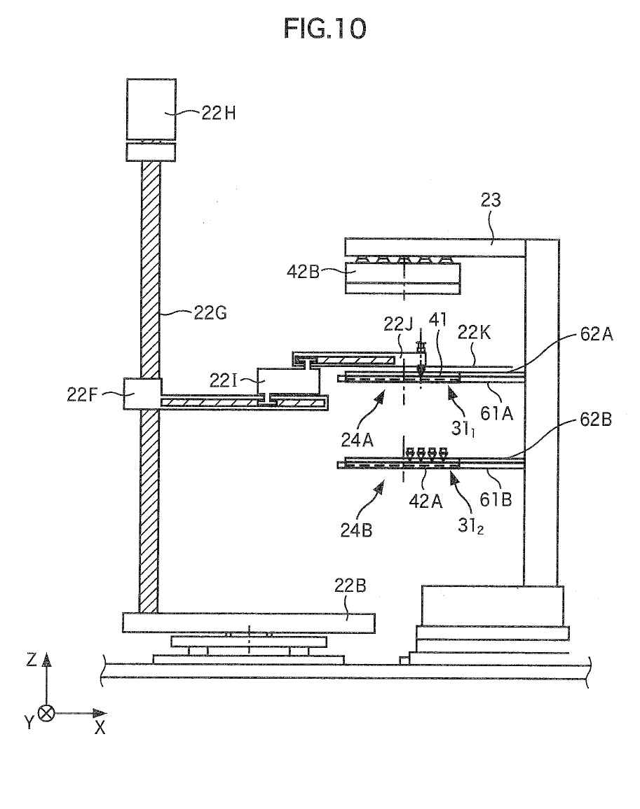

Subsequently, the transport unit 22 moves the dispenser 22L₁ to which the dispenser tip 51 is attached above the solution area 42A₃, and causes a solution such as a reagent to be aspirated from the aspiration port 53 with the dispenser tip 51. As shown in FIG. 10, after the dispenser 22L₁ is moved to above the culture vessel 41, which has been transported to the upper level of the tray holder 61A and fixed by the tray lug locking portion 62A, by moving the transport unit 22 in the positive direction of the Z axis, the solution aspirated by the disperser tip 51 is added to the culture vessel 41.

In addition, in the case solution remains in the dispenser tip 51 after adding solution, the dispenser 22L₁ is moved to above the waste liquid area 42A₄ of the vessel portion 42A fixed to the lower level of the tray holder 61B, the end of the dispenser tip 51 is inserted into the waste liquid port 54, and all of the waste liquid is expelled there from. Subsequently, the dispenser 22L₁ is further moved to above the used dispenser tip area 42A₂, and the used dispenser tip 51 is removed from the dispenser 22L₁.

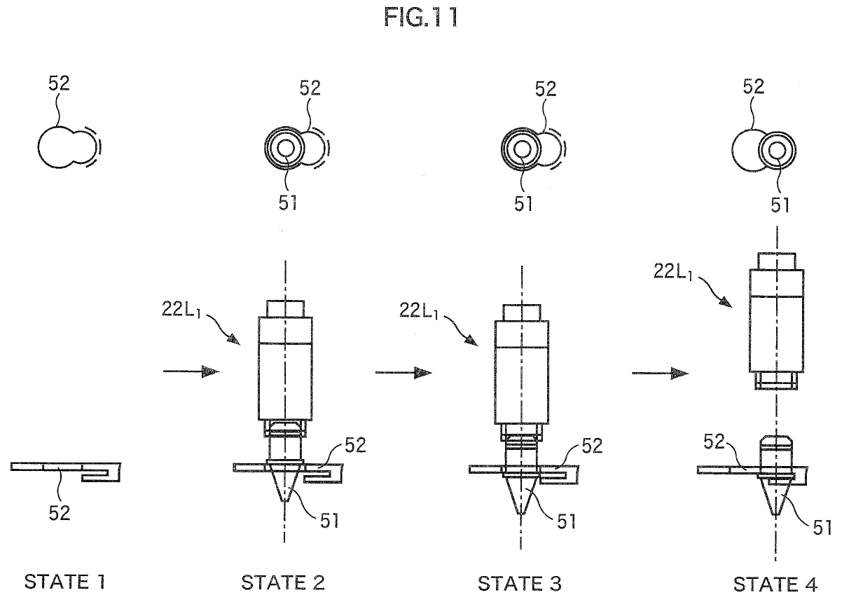

With respect to the method used to remove the dispenser tip 51, as indicated by “State 1” of FIG. 11, for example, the dispenser tip 51 is first inserted into the portion of a hole having a larger diameter among the holes 52 provided in the used dispenser tip area 42A₂ (12 holes 52 are provided in the example of FIG. 4) (“State 2” of FIG. 11). When a flange of the dispenser tip 51 has become lower than the upper plate of the used dispenser tip area 42A₂ of the vessel portion 42A (“State 3” of FIG. 11), the dispenser tip 51 moves to the portion of the hole having a smaller diameter. The dispenser tip 51 is then removed as a result of the dispenser 22L₁ being raised in the positive direction of the Z axis while in the above-mentioned state, causing the flange to catch on the lip of the hole 52 (“State 4” of FIG. 11).

Furthermore, although the explanation of the present embodiment uses an example of adding a solution to the culture vessel 41, in the case of, for example, preliminarily removing an amount of solution equal to the amount added prior to adding the solution, the above-mentioned operation is carried out after having attached the dispenser tip 51 placed in an insertion hole of the unused dispenser tip area 42A₁ to the dispenser 22L₂ in the same manner as the dispenser 22L₁, aspirating solution from the culture vessel 41, and then discarding the waste liquid in the waste liquid area 42A₄ of the vessel portion 42A.

Subsequently, once all dispensing work has been completed, the dispensing mode ends, and the tray lug locking portions 62A and 62B fixing the lugs 31C of the trays 31 are disengaged. Continuing, the transport unit 22 holds the lugs 31C of the tray 312 with the arm 22K and moves the tray 31₂ to the lid opening and closing unit 23, after which it conversely moves the vessel portion 42A in the positive direction of the Z axis and places the lid portion 42B suctioned by the suctioning portion 23A over the vessel portion 42A. Subsequently, in the case the tip solution storage vessel 42 that has been covered with the lid portion 42B is to be removed outside the incubator unit 11, the tip solution storage vessel 42 can be taken out through the access door 11A by carrying out the tip solution storage vessel 42 to the carrier unit 26 with the transport unit 22. In addition, the transport unit 22 carries out the culture vessel 41 on which dispensing work has been carried out from the dispenser unit 24A and stores it in the stocker unit 21.

As has been described above, according to the present invention, dispensing work in an incubator environment can be easily carried out by dispensing using the tip solution storage vessel 42 in which is provided respective areas for storing various types of elements required for realizing dispensing work.

In addition, according to the present invention, internal space can be used effectively making it possible to reduced the total size of the culture apparatus since the trays 31 on which are placed the culture vessel 41 and the tip solution storage vessel 42 can be transported to each mechanism within the incubator unit 11 by a single transport unit 22 instead of using a plurality of transport mechanisms.

Moreover, according to the present invention, since the transport unit 22 that transports the trays 31 on which are placed the culture vessel 41 and the tip solution storage vessel 42 has the dispensers 22L, it is able to carry out both transport work and dispensing work, and since it is not necessary to provide individual mechanisms for carrying out that work, the overall size of the culture apparatus can be reduced. In addition, since the transport unit 22 that transports the trays 31 on which are placed the culture vessel 41 and the tip solution storage vessel 42 is able to dispense at that location, dispensing work can be carried out rapidly.

Namely, although it had been conventionally required to provide separate transport means for the culture vessel 41 and the dispensers 22L, single transport means can now be shared by both. Moreover, as a result of being able to conserve space, the common transport mechanism can be installed within the incubator unit 11.

In addition, by making the size of the tip solution storage vessel 42 that houses unused dispenser tips 51, used dispenser tips 51, solution and waste liquid to be compatible with the size of the culture vessel 41 (having the size of a microwell plate), the tip solution storage vessel 42 can be transported over the transport path of the culture vessel 41. In addition, since the tip solution storage vessel 42 can be accessed with the access door 11A, it is not necessary to provide additional access doors in the bottom or sides of the incubator unit 11.

Furthermore, although the present embodiment was explained by using an example of the lugs 31C of the trays 31 being placed on the arm 22K when supporting the trays 31 on the arm 22K, a system may also be employed in which, for example, the trays 31 are clamped from the sides or a system in which the trays 31 are completely fixed and held using a magnet or air chuck and the like.

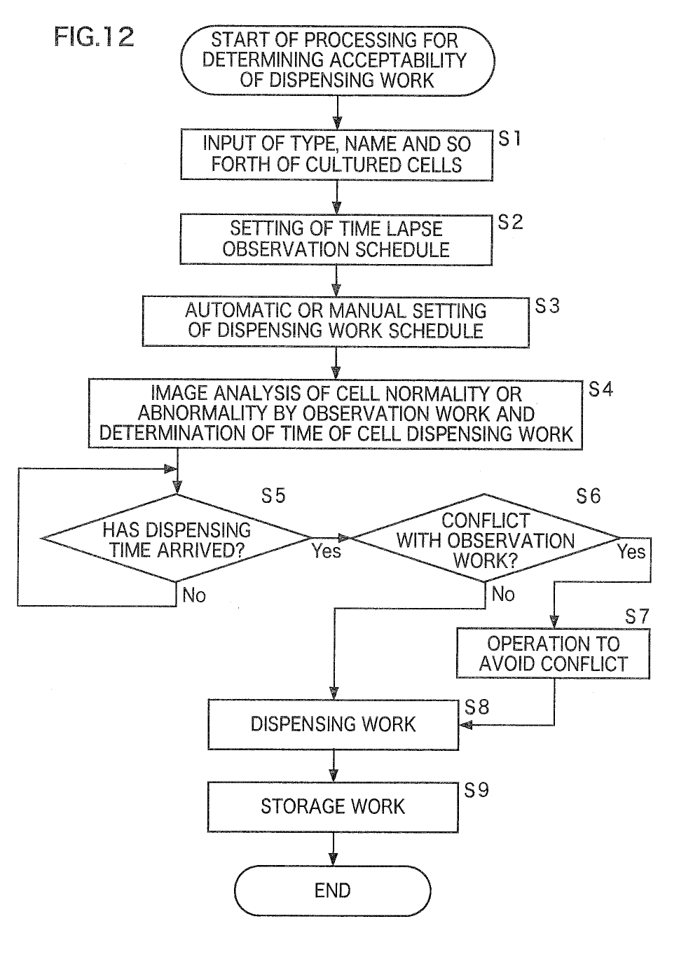

Next, an explanation is provided of another embodiment of the present invention with reference to the flow chart of FIG. 12.

The embodiment of FIG. 12 is basically based on the previously explained embodiment, and explanations of each component of the cell culture apparatus 1 are omitted.

In the cell culture apparatus 1 of FIG. 1, a microcomputer is contained in the control box 13, and the microcomputer governs various types of control such as control of the culture environment of the incubator unit 11 (such as the temperature to 37° C., the humidity to 90% and the oxygen concentration) to constant conditions, control of transport work by the transport unit 22 in order to perform transport work, storage work, dispensing work and observation work on the culture vessel 41, control of dispensing work by the dispensers 22L₁ and 22L₂ and the pump unit 27, and control of observation work by the observation unit 25 (composed of the illumination unit 25A and the CCD camera 25B).

Details of program control by the microcomputer of the control box 13 are shown in the flow chart of FIG. 12.

In addition, there are two types of images able to be acquired by the observation unit 25. The first is a bird's-eye view image in the form of a color image of the entire vessel, while the other is a microscopic image obtained with a microscope. The purpose of capturing the bird's-eye view image is to determine color changes in cell culture medium and obtain an image of the entire cell culture vessel. The microscopic image is realized with an object lens and an intermediate zoom optical system.

The cell culture apparatus 1 is an apparatus that stores a plurality of the culture vessels 41 in the stocker unit 21 while simultaneously being able to manage a plurality of different types of cultured cells or manage cultured cells corresponding to the respective experiment schedules of a plurality of users. The dispensers 22L₁ and 22L₂ and the pump unit 27 and the like required for dispensing work are housed within the cell culture apparatus 1 so as to be able to perform dispensing work on the cultured cells. Thus, since there may be conflicts in the time lapse schedule during ordinary cell culturing or in the observation schedule during dispensing work in the cell culture apparatus 1 during the course of management of culturing and dispensing work on different culture vessels being used by the same user or on a culture vessel 41A of one user and a culture vessel 41B of another user, it is necessary to avoid such conflicts.

The operations described in (1) to (3) below can be used as operations for avoiding the conflicts described above.
(1) One schedule is chronologically shifted to before or after the other schedule so that the dispensing work schedule does not interfere with the time lapse observation schedule.
(2) Dispensing work is performed directly by skipping the observation operation ordinarily performed as a part of dispensing work.
(3) An alarm indicating a conflict is emitted to inform a user.

Furthermore, the dispensing work here refers to, for example, medium replacement work, subculturing work, work involving dropping in reagents and the like, and in order to confirm in advance whether or not cultured cells are being cultured properly during that work, the culture vessels 41 are transported to the observation unit 25 and an assessment is made as to the growth status of the culture cells, and if the growth status thereof is determined to be normal, dispensing work is carried out following confirmation thereof. If growth status is not normal, the culture vessel 41 is discharged outside the culture apparatus.

The following provides a detailed explanation of the case of medium replacement as an example of dispensing work.

A color of the medium at the time the medium is to be replaced is stored in advance in a database for each of the cells. The culture vessel 41 containing cells is placed in a holder and transported from a carrier to the stocker unit 21. The culture vessel 41 is then transported from the stocker unit 21 to the observation unit 25 in accordance with an observation schedule. During the course of the observation schedule, a color bird's-eye view image of the entire vessel is always acquired. A comparison is then made between the color of the medium depicted in the bird's-eye view image and the color of the medium when it is to be replaced that has been pre-registered as data, and in the case a threshold value of the color of the medium when it is to be replaced has been exceeded, the apparatus determines that it is necessary to replace the medium. When it has been determined to replace the medium, the culture vessel 41 is carried to the dispensing area and the medium is replaced.

In addition, an observation operation (observation work) refers to, for example, transporting the culture vessel 41 to the observation unit 25 and assessing the growth status of cultured cells in order to periodically confirm growth status of cultured cells when culturing cells for an extended period of time, and if the growth status thereof is determined to be normal, the culture vessel 41 is moved to the stocker unit 21. If growth status is not normal, the culture vessel 41 is discharged outside the culture apparatus.

Next, an explanation is provided of program control of the above-mentioned avoidance operation along with control of the series of acceptance judgments made for dispensing work using the flow chart of FIG. 12.

In Step S1, the type and name of cells input by a user is accepted.

Namely, if the user is familiar with the cells, since a preliminarily prepared table in which dispensing times are stored (data table that correlates dispensing time data with types of cultured cells) is stored in the computer, the user simply enters the type and name of the cells.

In Step S2, time lapse observation schedules are set for each culture vessel 41 to carry out ordinary cell culturing (confirm cell growth status) by a single or a plurality of users.

The time lapse observation schedules can be set from an operating panel not shown of the cell culture apparatus 1, and more specifically, consists of setting time lapse conditions (including observation starting time, observation ending time, observation interval during the observation period and setting of observation points in the culture vessel 41) for each culture vessel 41. Cell culturing is begun in the culture vessels 41 in accordance with the time lapse observation schedules, image data of the cultured cells is acquired, and a judgment is subsequently made as to whether or not dispensing work is to be performed.

Furthermore, these time lapse observation schedules are programmed and controlled independently for each work flow.

In Step S3, a dispensing work schedule is set if there is a “dispensing time storage table” that matches the cell type and name input in Step S1. However, since the user sets the dispensing work schedule manually in the case a matching storage table does not exist, input for setting the dispensing work schedule is accepted.

Image analysis of cell normality or abnormality by observation work as well as a determination as to the time when cell dispensing work is to be performed are performed in Step S4.

Namely, images of the cultured cells (sample) are periodically captured by the observation unit 25 in accordance with the time lapse observation schedule set in Step S2 and image data for the cultured cells is accumulated until the dispensing time determined with the dispensing work schedule of Step S3 has arrived. Growth status of the cells, namely whether or not the cells are growing normally or abnormally, is assessed by analyzing the cell image data.

In addition, a determination is made as to whether or not the time at which the cells are to be dispensed coincides with a dispensing time in the storage table of Step S1 based on an analysis of the cell image data. If the time discrepancy with the dispensing time in the storage table is within a prescribed time range, the dispensing time in the storage table is used. However, if the time discrepancy is outside the prescribed time range, a dispensing time determined from the cell image analysis data is used, and the dispensing time of subsequent dispensing work in the storage table is revised.

The microcomputer monitors whether or not the cultured cell dispensing time has arrived, and in the case the dispensing time has been determined to have arrived in Step S5, processing proceeds to Step S6. The cultured cells are continued to be observed in accordance with the time lapse observation schedule until the dispensing time arrives.

In Step S6, a determination is made as to whether or not a conflict has occurred in observation work.

Namely, a determination of the acceptability of dispensing work is always carried out by first preferentially performing observation work prior to the dispensing work. Here, a problem occurs if there is a conflict between observation work of the dispensing work schedule and observation work of the time lapse observation schedule. For example, in the case the culture vessel 41A on which observation work of the dispensing work schedule is to be performed differs from the culture vessel 41B on which observation work according to the time lapse observation schedule is to be performed, observation work cannot be performed on the two culture vessels at the same time.

In the case a conflict has occurred in observation work in Step S6, processing proceeds to Step S7.

In Step S7, an operation for avoiding a conflict in observation work is carried out. An operation previously described in (1) to (3), for example, can be carried out to avoid a conflict in such cases. Firstly, one of the schedules is chronologically shifted to before or after the other schedule to prevent interference between the time lapse observation schedule and the dispensing work schedule. Secondly, observation work accompanying dispensing work is skipped to enable dispensing work to be performed directly. Thirdly, an alarm indicating the occurrence of a conflict is emitted to inform a user. Conflicts between observation work are avoided by the above-mentioned operations.

Dispensing work is performed in Step S8 after conflicts have been avoided with the above-mentioned operations.

On the other hand, in the case a conflict has been determined to not exist in observation work in Step S6, observation work is performed according to the dispensing work schedule in Step S8. A determination is then made by the observation work as to whether the growth status of cultured cells immediately prior to dispensing work is normal or abnormal. Although dispensing work is allowed to proceed in the case growth status has been determined to be normal, in the case of having been determined to be abnormal, the culture vessel 41 is discharged outside the culture apparatus without proceeding to dispensing work.

In this manner, the embodiment of FIG. 12 is composed so that a determination as to the acceptability of dispensing work is made to prevent unnecessary dispensing work from being performed.

When dispensing work is completed in Step S8, storage work is performed in Step S9 in which the culture vessel 41 is returned to the stocker unit 21 followed by continuation of culturing.

Although the present embodiment as described above is explained as using a portion of a transport apparatus as an apparatus that carries out dispensing work, the present embodiment is not limited thereto, but rather, for example, a dedicated dispenser may also be installed within the culture apparatus.

Furthermore, in the present description, steps for describing the program naturally not only include processing carried out chronologically in a described order, but also include processing carried out in parallel or individually that is not necessarily carried out in chronological order.

In addition, embodiments of the present invention are no limited to the above-mentioned embodiments, but rather various modifications can be made thereto within a range that does not deviate from the gist of the present invention.

1. A culture apparatus that houses a culture vessel containing a sample, and has an internal space maintained and managed at prescribed culture environment conditions, to culture the sample,
wherein the internal space is provided with:
a dispensing area for dispensing into the culture vessel;
an observation area for arranging observation means for observing the sample placed in the culture vessel through an observation optical system; and
a transport area for arranging transport means for transporting the culture vessel in the horizontal and vertical directions,
the dispensing area and the observation area are respectively arranged adjacent to the transport area in the horizontal direction which is a direction of transport of the transport means, and
the transport means transports the culture vessel between the dispensing area and the observation area in order to carry out dispensing work in the dispensing area and observation work in the observation area.

2. The culture apparatus according to claim 1, wherein
the internal space further has a storage area adjacent to the transport area, for arranging storage means having storage shelves for storing a plurality of the culture vessels,
the storage means is arranged on one side of the lengthwise direction of the transport area while the dispensing area and the observation area are arranged on the other side thereof, and
the transport means transports the culture vessel to the storage means in the storage area following completion of the dispensing work and the observation work.

3. The culture apparatus according to claim 1, wherein
an aspiration unit is disposed in the transport means, and a dispenser tip for aspirating a prescribed solution to the aspiration unit and dispensing the solution into the culture vessel is attached to the transport means, and
the transport means transports the culture vessel to the dispensing area and dispenses into the culture vessel with the dispenser tip.

4. The culture apparatus according to claim 3, wherein
the dispensing area is an area where dispensing work such as replacement of culture liquid of the culture vessel or injection of a reagent is carried out, and a mounting shelf is formed therein on which the culture vessel is placed for dispensing work.

5. The culture apparatus according to claim 4, wherein
in the case the culture vessel has a lid, removal means for removing the lid of the culture vessel by suctioning is arranged on an upper part of the mounting shelf in the dispensing area.

6. A culture apparatus that houses a culture vessel containing a sample, and has an internal space maintained and managed at prescribed culture environment conditions, to culture the sample, the culture apparatus comprising:
the internal space in which is formed a storage area for arranging storage means having storage shelves for storing a plurality of the culture vessels, a dispensing area for dispensing into the culture vessels, an observation area for arranging observation means for observing the sample placed in the culture vessels through an observation optical system, and a transport area for arranging transport means for transporting the culture vessels in the horizontal and vertical directions;
the transport means for transporting the culture vessels between the storage area, the dispensing area and the observation area; and
control means for controlling transport work of the transport means for transporting the culture vessels between the dispensing area, the observation area and the storage area, dispensing work in the dispensing area, observation work in the observation area and storage work in the storage area,
wherein the control means causes the observation means to perform the observation work prior to the dispensing work, determines whether or not the dispensing work is to be performed based on image data of the sample acquired during the observation work, and either controls transport so that the culture vessels are transported from the observation area to the dispensing area, or controls transport so that the culture vessels are transported from the observation area to the storage area.

7. The culture apparatus according to claim 6, wherein the control means causes the observation means to perform the observation work prior to the dispensing work, analyzes growth status of the sample based on image data of the sample acquired during the observation work, determines the time at which the dispensing work is to be performed, returns the culture vessels to the storage shelves of the storage area until that time arrives, and controls transport so that the culture vessels are transported from the storage area to the dispensing area when the dispensing time arrives.

8. The culture apparatus according to claim 6, wherein
the control means includes a storage table of dispensing work times based on the type of cultured cells of the sample, and
the control means causes the observation means to perform the observation work prior to the dispensing work at the timing of the dispensing work times in the storage table, determines whether or not the dispensing work is to be performed based on image data of the sample acquired during the observation work, and either controls transport so that the culture vessels are transported from the observation area to the dispensing area, or controls transport so that the culture vessels are transported from the observation area to the storage area.

9. The culture apparatus according to claim 6, wherein
the control means accepts a time lapse observation schedule for the sample, and controls transport so that the culture vessels are transported from the storage area to the observation area in accordance with the time lapse observation schedule, and
the control means analyzes growth status of the sample based on image data of the sample acquired during observation work in accordance with the time lapse observation schedule, determines the time at which the dispensing work is to be performed, and sets a dispensing work schedule for the dispensing work in the time lapse observation schedule.

10. The culture apparatus according to claim 9, wherein
when there is a conflict between the time lapse observation schedule and the dispensing work schedule, the control means either gives priority to work of either one of the schedules, or shifts the dispensing work schedule before or after the time lapse observation schedule.

11. The culture apparatus according to claim 6, wherein
the control means accepts a time lapse observation schedule for the sample, and together with controlling transport so that the culture vessels are transported from the storage area to the observation area in accordance with the time lapse observation schedule, sets a dispensing work schedule for the dispensing work in the time lapse observation schedule, and
when there is a conflict between the time lapse observation schedule and the dispensing work schedule, the control means gives priority to work of either one of the schedules, or shifts the dispensing work schedule before or after the time lapse observation schedule.

12. The culture apparatus according to claim 10, wherein
when there is a conflict between observation work included in the time lapse observation schedule of a first culture vessel and observation work included in the dispensing work schedule of a second culture vessel, the control means gives priority to observation work of the time lapse observation schedule and skips observation work of the dispensing work schedule.

13. The culture apparatus according to claim 11, wherein
when there is a conflict between observation work included in the time lapse observation schedule of a first culture vessel and observation work included in the dispensing work schedule of a second culture vessel, the control means gives priority to observation work of the time lapse observation schedule and skips observation work of the dispensing work schedule.
